(19) United States
(12) Patent Application Publication
(10) Pub. No.: US 2008/0035146 A1
(43) Pub. Date: Feb. 14, 2008

(54) METHODS, SYSTEMS AND COMPUTER PRODUCTS FOR FILLING LUNGS

(76) Inventor: Jerry Crabb, Buford, GA (US)

Correspondence Address: CANTOR COLBURN, LLP, 55 GRIFFIN ROAD SOUTH, BLOOMFIELD, CT, 06002, US

(21) Appl. No.: 11/745,095

(22) Filed: May 7, 2007

Related U.S. Application Data
(60) Provisional application No. 60/798,047, filed on May 5, 2006.

(51) Int. Cl. A61M 16/00; A62B 7/00

(52) U.S. Cl. 128/204.18; 128/204.23; 600/538

(57) ABSTRACT

Methods, systems, and computer products for filling lungs. Exemplary embodiments include a method for filling lungs, the method including identifying an ailment for active lung filling treatment, determining a preset volume to fill into the lungs, determining an inhalation period and determining an exhalation period.

Priority based on U.S. Provisional Patent Application. Ser. No. 60/798,047, filed May 5, 2006, and entitled, “LUNG FILLING METHODS”, is claimed, and the content of which is incorporated herein by reference in its entirety.

### BACKGROUND OF THE INVENTION

1. Field of the Invention

The present invention relates generally to the field of breathing and cardiac ailments and more particularly methods, systems, and computer products for filling kings.

2. Description of Background

In general, there exist several ailments that affect the breathing and cardiac systems of a patient. In addition, the ailments can be aggravated by poor breathing habits. For example, congestive heart failure can affect a patient's breathing. However, it has been, determined that deep breathing can alleviate congestive heart failure. However, is the patient is a shallow breather, the shallow breathing can aggravate the congestive heart failure.

While present breathing aid machines typically provide a constant airflow through nasal tubing or through masks to provide oxygen to patients, the breathing aid machines do not otherwise aid and teach patients to breath more effectively, such as by taking deeper breaths. As such, present breathing aid machines are passive. Other passive devices are used for training people to breathe by breathing through, the device that restricts breathing thereby causing the person to work harder to breathe. However, such passive devices do not actively aid a patient in breathing cycles.

There persists a lack of breathing methods and systems that actively aid persons with breathing ailments in both inhalation and exhalation cycles.

### SUMMARY OF THE INVENTION

Exemplary embodiments include a method for filling lungs, the method including identifying an ailment, for active lung filling treatment, determining a preset volume to fill into the lungs, determining an inhalation period and determining an exhalation period.

Additional embodiments include a method for treating a breathing ailment, the method including identifying an automated breathing regimen and providing a breathing machine configured to implement the breathing regimen

Further embodiments include a system for filling lungs, the system including a controller, a breathing machine coupled to the controller and having a process to identify an ailment for active lung filling treatment, determine a preset volume to fill into the lungs, determine an inhalation period and determine an exhalation period.

System and computer program products corresponding to the above-summarized methods are also described and claimed herein.

Additional features and advantages are realized through the techniques of the present invention. Other embodiments and aspects of the invention are described in detail herein and are considered a part of the claimed invention. For a better understanding of the invention with advantages and features, refer to the description and to the drawings.

### TECHNICAL EFFECTS

As a result of the summarized invention, technically methods and systems for providing active breathing regimens have been, achieved.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates a flowchart of a lung filling method in accordance with exemplary embodiments; and

FIG. 2 illustrates a system diagram of an exemplary lung-filling system.

The detailed description explains the preferred embodiments of the invention, together with advantages and features, by way of example with reference to the drawings.

### DETAILED DESCRIPTION OF THE INVENTION

In general the following description discusses embodiments and implementations for assisted and unassisted, as well as monitored and adjusted breathing methods to address a variety of breathing conditions, particularly during sleeping hours.

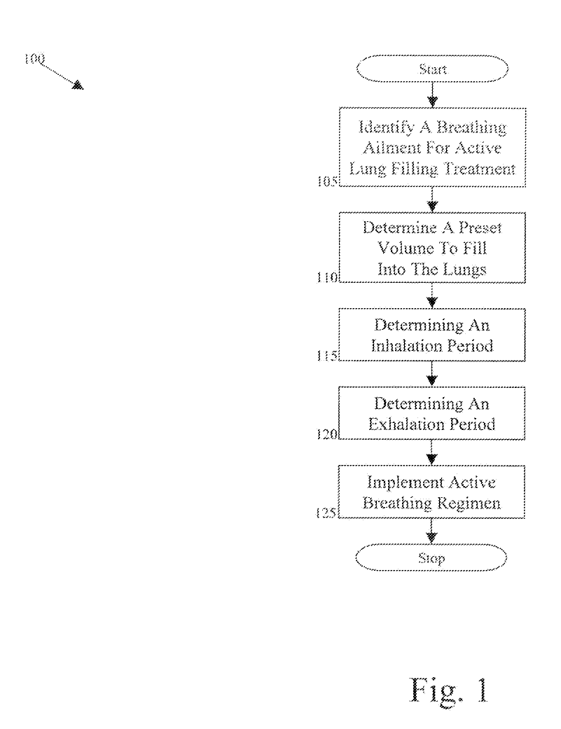

Turning now to the drawings in greater detail, FIG. 1 illustrates a flowchart of a lung filling method 100 in accordance with exemplary embodiments.

In accordance with a first method for filling lungs, the method 100 can include various steps, typically identified and defined by an appropriate health care practitioner to aid a patient in managing or even overcoming a breathing condition or ailment. The method 100 can therefore include identifying an ailment for which active lung tilling is required or desired at step 105. The method 100 can further include determining a preset volume to fill into the lungs at step 110. It is appreciated that different patients have different lung capacities. As such, a health care practitioner can determine the patient's lung capacity in accordance with exemplary embodiments. By identifying the lung capacity, the health practitioner can determine a preset volume that can be actively filled into the patient's lungs. Furthermore, the healthcare practitioner can determine an inhalation period at step 115. The inhalation period can be implemented to actively fill the patient's lungs for the inhalation period to the preset lung capacity determined at step 110. At step, 120, an exhalation period can be determined.

In exemplary embodiments, due to increased exhalation, levels of carbon dioxide, bacteria and viruses, along with contaminants delivered by the lungs to the exhausted air coupled to a breathing machine, thereby reduces the levels of carbon dioxide, bacteria and viruses, and contaminants in the patient's bloodstream.

In exemplary embodiments, methods can further include applying an air pressure through at least one of the nose, the mouth and a combination of the nose and mouth. It is appreciated that several types of readily available masks and other breathing aid apparatuses can be implemented as necessary or as defined by die health care provider.

Exemplary embodiments can include further filling the lungs via the at least one of the nose, the mouth, and a combination of the nose and the mouth to the preset volume for the preset inhalation period. It is generally appreciated that the preset volume can match or exceed the patient's lung volume or ideal volume that would be attainable but for the presence of the lung condition or ailment.

Exemplary embodiments can further include allowing the lungs to exhale for the exhalation period after the present volume has been attained. In general, it is contemplated that the patient exhales on his or her own breathing power and pressure, it is contemplated that in other exemplary embodiments, a vacuum, or other reduced pressure methods and systems can be applied in order to better assist or even pull an exhalation from the patient to even further assist in the patient's breathing.

Exemplary embodiments can further include providing a backpressure against exhalation pressure associated with the exhalation period. In this implementation, it is contemplated that a patient is forced to breathe harder and stronger against a backpressure which in turn trains the patient to unconsciously breathe better and more efficiently. In general, in a typical implementation, the exhalation pressure is greater than the backpressure. In such an implementation it is appreciated that if the backpressure is too great then even strong breathers may not be able to exhale against the backpressure. However, it is contemplated that as the patient becomes a more efficient breather, the backpressure can be increased and possibly exceed the exhalation pressure if the patient can provide a stronger exhalation pressure. The method can generally include monitoring the backpressure so that it does not exceed the exhalation pressure.

Exemplary embodiments can further include providing a seal between the nose, mouth and a combination of the nose and the mouth region and an external environment. In this implementation, the seal ensures that there is no leakage or other airflow other than the inhalation and exhalation flows to help to patient experience assisted and unassisted breathing by way of the method only.

Exemplary embodiments can further include matching the preset volume to the inhalation period such that the inhalation period provides air pressure long enough to fill the preset volume.

Exemplary embodiments can further include monitoring a breathing pattern and providing a predetermined automated breathing pattern if the breathing pattern has decreased, in this way, the method can be monitored based on, for example, expected normal and healthy breathing patterns, in accordance with accepted, medical guidelines. If it is determined that an abnormal or unhealthy breathing pattern is detected, then steps can be taken to assist in breathing as much as necessary to protect, normal breathing patterns.

Exemplary embodiments can further include providing a breathing pathway into a patient's lungs. In general, it is contemplated that pathways can be determined not only to help determine breathing regimens but to also advantageously administer medication, if determined. Therefore, the method can further include identifying a medication for placement into the pathway for application into the patient's lungs and introducing the medication into the pathway.

In another advantageous method for treating a breathing ailment, several other types of steps can include identifying an automated breathing regimen and providing an active breathing machine configured to implement the breathing regimen.

Exemplary embodiments can further include correlating the breathing regimen with a known condition, wherein the known breathing condition affects normal function of a patient's lungs.

It is appreciated that there are many types of breathing conditions and ailments that inflict patients. In general, the conditions of most concern are those that occur in the sleeping hours that can affect a patient, particularly those patients who are older and may not have the physical ability to unconsciously compensate for the condition. These conditions can include but are not limited to emphysema, congestive heart failure, sleep apnea and the like.

In implementing the method, identifying an automated breathing regimen, can include measuring a lung volume of a patient, determining a volume of air to match or exceed the lung volume and defining an inhalation period required to match or exceed the lung volume.

Exemplary embodiments can therefore further include determining a rate of airflow correlated with the inhalation period, and providing airflow into the patient's lungs at the rate of airflow for the inhalation period.

In addition, similar to as described above, exemplary embodiments can further include defining an exhalation period required to allow the volume applied to the lungs to empty the lungs under the patient's unassisted breathing pressure associated with unassisted exhalation.

In addition, exemplary embodiments can further include removing the air flow to allow the patient to empty the lungs under the unassisted breathing pressure, measuring the unassisted breathing pressure and applying a counter pressure against the unassisted breathing pressure.

In another embodiment, implementations can include varying the programs to test the normal breathing of a patient such as for example, periodically removing assisted inhalation, and measuring inhalation depth to determine if a pre-determined lung volume has been attained.

Exemplary embodiments can further include providing assisted inhalation if the pre-determined lung volume has not been met or exceeded, measuring exhalation pressure and applying a pressure against the inhalation pressure to force further exhalation pressure.

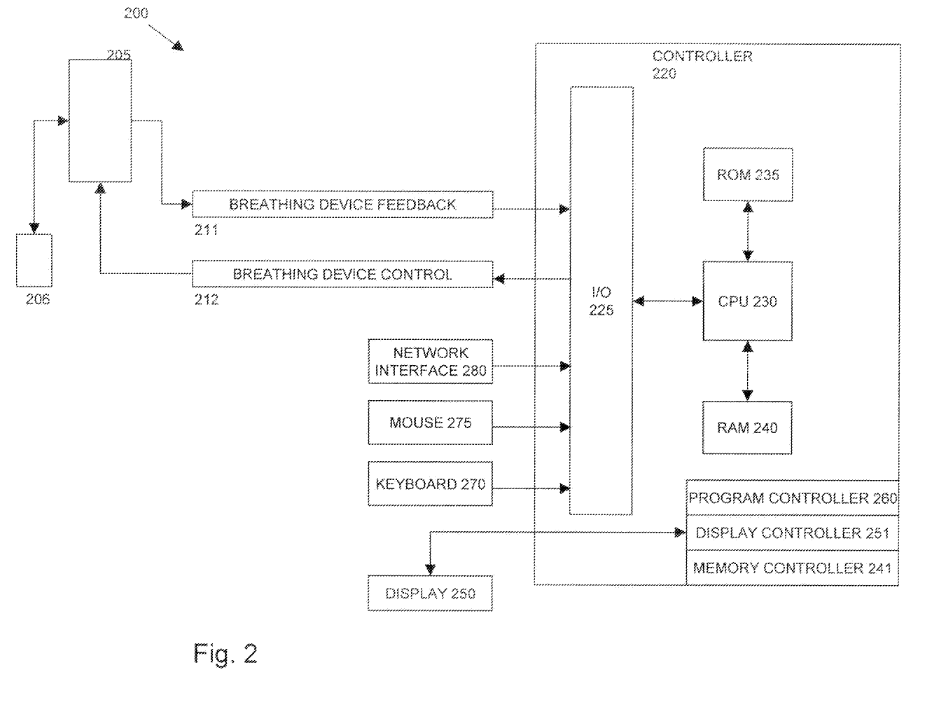

In general, the active breathing machine configured to implement the breathing regimen is a programmable breathing machine. FIG. 2 illustrates a system diagram of an exemplary lung-filling system 200.

In exemplary embodiments, the system 200 includes a breathing machine 205 configured to provide active air filling to a patient's lungs to a preset capacity for inhalation and exhalation periods as discussed above. A delivery device 206, such as a mask, is coupled to the breathing machine 205 to interface with the patient, via the nose, mouth, combination of the nose and the mouth, and/or lungs as described above. Pathways, such as to deliver medication, can be provided via the breathing machine 205 and delivery device 206. The system can further include a breathing device feedback pathway 211 and a breathing device control pathways 212 coupled to a controller 220, via an I/O interface 225. In exemplary embodiments, the breathing device feedback pathway 211 is configured to provide patient breathing patterns to the controller 220 such that the controller 220 can provide adjustments to the identified breathing regimen, if required. In further exemplary embodiments, the breathing device control 212 is configured to provide commands to the breathing machine 205, including, but not limited to lung capacity data, inhalation period data, exhalation period data, etc.

The controller 220 may thus collect and provide various signals, such as from the breathing device feedback pathway 211 and the breathing device control pathway 212, respectively, to control the breathing machine 205. The controller 220 may execute computer readable instructions for functions such as breathing control logic, breathing pattern logic, etc., which may be embodied as computer program products, in exemplary embodiments, the controller 220 includes a central processing unit (CPU) 230, a read-only memory (ROM) 235 (e.g., non-volatile EEPROM), and a volatile memory such, as a random access memory (RAM) 240. The CPU 230 operably communicates with the ROM 235, the RAM 240, and the I/O Interface 2225. Computer readable media including the ROM 235 and the RAM 240 may be implemented using any of a number of known memory devices such as PROMs, EPROMs, EEPROMS, flash memory or any other electric, magnetic, optical or combination memory device capable of storing data, some of which represent executable instructions used by the CPU 230. In exemplary embodiments, the CPU 230 communicates via the I/O interface 225 with the position the breathing device feedback pathway 211, which can include sensing the state of the breathing machine 205. The CPU can further communicate via the I/O interlace 225 with the breathing device control pathway 212 to make any necessary adjustments to the active breathing regimen, if necessary. In exemplary embodiments, the adjustments made to the active breathing regimen can be in response to feedback data received form the breathing device feedback pathway 211. While the breathing device feedback pathway 211 and the breathing device control pathway 212 are depicted as separate inputs to the I/O interface 225, the signals may be otherwise coupled, packetized, or encoded.

Referring still to FIG. 2, the system 200 can further include other combinations of controllers, including but not limited to a memory controller 241 configured to control memory operations of the ROM 235 and the RAM 240, a display controller 251 coupled to a display 250, and configured to control display operations of the display 250, and a program controller 260 configured to control the operations of various program instructions as discussed further below. The system 200 can further include a combination of computer peripheral devices coupled to the controller 220 including but not limited to input devices such as a keyboard 270 and a mouse 275, a network interface 280, etc. The network interface 280 can allow a practitioner to control the breathing machine remotely, if necessary.

In exemplary embodiments, it is appreciated that various algorithms can be programmed in to the controller 220 for causing the breathing machine 205 to implement active breathing regimens for a patient. Such algorithms can be stored in both the ROM 235 and RAM 240 as necessary in accordance with exemplary embodiments. The algorithms can be pre-written to correspond to specific breathing ailments, thereby providing a health practitioner a starting point to initiate an active breathing regimen. The health practitioner can adjust the algorithms according to the specific patient's lung capacity, ability to inhale and exhale, etc. The ROM 235 and ROM 240 can also store modifications to the algorithms for use by the breathing machine 205. As mentioned above, the program controller 260 can control algorithm functions, which can include any operations and sub-operations of the algorithm. Accordingly, ROM 235 and RAM 240 can store a plurality of algorithms having a plurality of operations and sub-operations, controlled by the program controller 260. Each algorithm in the plurality of algorithms can be called by the controller 220 to perform an operation or sub operation of the methods described herein.

It is appreciated that the embodiments of the methods described herein can be implemented in many conventional as well as any proprietary breathing machines, devices and apparatuses. It is further generally appreciated that the methods can be advantageously programmable into the aforementioned machines, devices and apparatuses. Thus the methods can be implemented in software firmware and the like and can be adjusted as desired or necessary to cater to individual patients and conditions.

The software techniques and methods discussed above can be implemented in digital electronic circuitry, or in computer hardware, firmware (as discussed), software, or in combinations of them. Apparatus may be implemented in a computer program product tangibly embodied in a machine-readable storage device for execution by a programmable processor; and methods may be performed by a programmable processor executing a program of instructions to perform functions by operating on input data and generating output. Further embodiments may advantageously be implemented in one or more computer programs that are executable on a programmable system including at least one programmable processor coupled to receive data, and instructions from, and transmit data and instructions, to a data storage system, at least one input device, and at least one output device. Each computer program may be implemented in machine language or assembly language which can be assembled or translated, or a high level procedural or object-oriented programming language, which can be complied or interpreted. Suitable processors include, by way of example, both, general and special purpose microprocessors. Generally, a processor receives instructions and data, from read-only memory and or RAM. Storage devices suitable for tangibly embodying computer program instructions and data include all forms of non-volatile memory, including by way of example semiconductor memory devices, such as EPROM, EEPROM, and flash memory devices, magnetic disks such as internal hard disks and removable disks; magneto-optical disks; and CD-ROM disks. Any of the foregoing may be supplemented by, or incorporated in, specially designed application specific integrated circuits (ASICs).

The capabilities of the present invention can be implemented in software, firmware, hardware or some combination thereof.

As one example, one or more aspects of the present invention can be included in an article of manufacture (e.g., one or more computer program products) having, for instance, computer usable media. The media has embodied therein, for instance, computer readable program code means for providing and facilitating the capabilities of the present invention. The article of manufacture can be included as a part of a computer system or sold separately.

Additionally, at least one program storage device readable by a machine, tangibly embodying at least one program of instructions executable by the machine to perform the capabilities of the present invention can be provided.

The flow diagrams depicted herein are just examples. There may be many variations to these diagrams or the steps (or operations) described therein without departing from the spirit of the invention. For instance, the steps may be performed in a differing order, or steps may be added, deleted or modified. All of these variations are considered a part of the claimed invention.

The foregoing description and drawings comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions and the associated drawings. Although specific terms may be employed herein, they are used in a generic and descriptive sense only and not for purposes of limitation. Accordingly, the present invention is not limited to the specific embodiments illustrated herein, but is limited only by the following claims.

What is claimed is:

1. A method for filling lungs, the method comprising:
identifying an ailment, for active lung tilling treatment;
determining a preset volume to fill into the lungs;
determining an inhalation period; and
determining an exhalation period.

2. The method as claimed in claim 1, further comprising applying an air pressure through at least one of the nose, the mouth, and a combination of the nose and the mouth.

3. The method as claimed in claim 2 further comprising filling the lungs via the at least one of the nose, the mouth, and a combination of the nose and the mouth to the preset volume for the preset inhalation period.

4. The method as claimed in claim 1 further comprising allowing the lungs to exhale for the exhalation period after the present volume has been attained.

5. The method as claimed in claim 4 further comprising applying a backpressure against exhalation pressure associated with the exhalation period.

6. The method as claimed in claim 5 wherein the exhalation pressure is greater than the backpressure.

7. The method as claimed in claim 5 further comprising monitoring the backpressure so that it does not exceed the exhalation pressure.

8. The method as claimed in claim 1, further comprising providing a seal between the nose, mouth and a combination of the nose and mouth region, and an external environment.

9. The method as claimed in claim 1 further comprising matching the preset volume to the inhalation period such that the inhalation period provides air pressure long enough to nil the preset volume.

10. The method as claimed in claim 1 further comprising monitoring a breathing pattern.

11. The method as claimed in claim 10 further comprising providing a predetermined automated breathing pattern if the breathing pattern has decreased.

12. The method as claimed in claim 1 further comprising providing a breathing pathway into a patient's lungs.

13. The method as claimed in claim 12 further comprising identifying a medication for placement into the pathway for application into the patient's lungs.

14. The method as claimed in claim 13 further comprising introducing the medication into the pathway.

15. A method for treating a breathing ailment, the method comprising:
identifying an automated breathing regimen; and
providing means for implementing the breathing regimen

16. The method as claimed in claim 15 further comprising correlating the breathing regimen with a known condition.

17. The method as claimed in claim 16 wherein the known breathing condition affects normal function of a patient's lungs.

18. The method as claimed in claim 17 wherein the condition is selected from the group consisting of: emphysema, congestive heart failure and sleep apnea

19. The method as claimed in claim 15 wherein identifying an automated breathing regimen, comprises:
measuring a lung volume of a patient;
determining a volume of air to match or exceed the lung volume; and
defining an inhalation period required to match or exceed the lung volume.

20. The method as claimed in claim 19 further comprising determining a rate of airflow correlated with the inhalation period.

21. The method as claimed in claim 20 further comprising providing airflow into the patient's lungs at the rate of airflow for the inhalation period.

22. The method as claimed in claim 19 further comprising defining an exhalation period required to allow the volume applied to the lungs to empty the lungs under the patient's unassisted breathing pressure associated with unassisted exhalation.

23. The method as claimed in claim 22 further comprising removing the airflow to allow the patient to empty the lungs under the unassisted breathing pressure.

24. The method as claimed in claim 23 further comprising measuring the unassisted breathing pressure.

25. The method as claimed in claim 24 further comprising applying a counter pressure against the unassisted breathing pressure.

26. The method as claimed in claim 15 further comprising periodically removing assisted inhalation and measuring inhalation depth to determine if a pre-determined lung volume has been attained.

27. The method as claimed in claim 26 further comprising providing assisted inhalation if the pre-determined lung volume has not been met or exceeded.

28. The method as claimed in claim 27 further comprising measuring exhalation pressure.

29. The method as claimed in claim 28 further comprising applying a pressure against the inhalation pressure to force further exhalation pressure.

30. The method as claimed in claim 15 wherein the means for implementing the breathing regimen is a programmable breathing machine.

31. A system for tilling lungs, the system comprising:
a controller;
a breathing machine coupled to the controller and having a process to:
identify an ailment for active lung filling treatment;
determine a preset volume to fill into the lungs;
determine an inhalation period; and
determine an exhalation period.

32. The system as claimed in claim 31, wherein the exhalation period provides increased exhalation, thereby reducing levels of carbon dioxide, bacteria and viruses, along with contaminants delivered by lungs to exhausted air coupled to the breathing machine, thereby reducing the levels of carbon dioxide, bacteria and viruses, and contaminants in a patient's bloodstream.
